(19) United States
(12) Patent Application Publication
(10) Pub. No.: US 2014/0052044 A1
(43) Pub. Date: Feb. 20, 2014

(54) APPARATUS AND METHOD FOR VENTING GAS FROM A LIQUID

(71) Applicants: Martin Joseph Crnkovich, Walnut Creek, CA (US); Colin Weaver, Pleasanton, CA (US)

(72) Inventors: Martin Joseph Crnkovich, Walnut Creek, CA (US); Colin Weaver, Pleasanton, CA (US)

(21) Appl. No.: 14/009,243

(22) PCT Filed: Mar. 29, 2012
(86) PCT No.: PCT/US12/31153

§ 371 Date: Oct. 1, 2013

Related U.S. Application Data
(60) Provisional application No. 61/470,680, filed on Apr. 1, 2011.

(51) Int. Cl. A61M 1/16; B01D 19/00

(52) U.S. Cl. CPC A61M 1/16; B01D 19/0063
USPC 604/6.09; 137/199; 137/1

(57) ABSTRACT

The present invention relates to an apparatus and method for venting a gas from a liquid, which are applicable to a wide variety of medical liquid delivery systems. The apparatus includes level detectors, a clamp, and a control apparatus operably connected to the level detectors and the clamp. The method includes detecting whether liquid or gas is present at the level detectors, and opening and closing a clamp to vent gas.

### RELATED APPLICATION

This application claims the benefit of U.S. Provisional Application No. 61/470,680, filed on Apr. 1, 2011.

The entire teachings of the above application are incorporated herein by reference.

### BACKGROUND OF THE INVENTION

Hemodialysis is the diffusive transfer of small solutes out of blood plasma by diffusion across a semi-permeable membrane. Dialysis proceeds due to a concentration gradient across the membrane such that solutes diffuse from a liquid having a higher concentration to a liquid having a lower concentration. Hemodialysis removes toxic substances and metabolic waste from the bloodstream using an extracorporeal circuit with components designed to perform ultrafiltration and diffusion on the blood. Before the blood is returned to the body, air bubbles are removed from the blood to inhibit embolisms.

Gas venting chambers for hemodialysis systems have been disclosed in the art. For example, a conventional system is disclosed in U.S. Publication No. 2007/0106198, which describes a chamber for use in an extracorporeal liquid system. The conventional system includes a microporous filter at the top of the chamber that allows gas in the liquid to vent from the chamber. In such a system, it is important to minimize contact between the liquid (e.g., blood) and the microporous filter. Should the blood contact the filter, proteins present within the blood can be deposited on the filter, thus clogging the filter and decreasing the ability of gas (e.g., air) to exit through the filter.

Despite the fact that there are existing systems for venting a gas from a liquid, there is a need for improved systems that are reliable, affordable, and simple to use in either a clinical setting or in the home. In particular, there is a need for an apparatus and method for venting gas from a liquid that prevents the liquid in the gas separation chamber from contacting a hydrophobic membrane covering the outlet where the gas vents to the atmosphere.

### SUMMARY OF THE INVENTION

The present invention relates to a gas venting apparatus and method, which are applicable to a wide variety of medical liquid delivery systems. The embodiments discussed below, however, are directed generally to dialysis, such as hemodialysis (“HD”) and peritoneal dialysis (“PD”).

In one embodiment of this invention, an apparatus for venting gas contained in a liquid flowing in a liquid flow circuit includes a gas collection chamber located within the liquid flow circuit so that liquid flows through the chamber allowing gas to separate from the liquid and establish a gas-liquid interface within the chamber. A gas vent chamber is provided at the top of the gas collection chamber through which gas within the chamber can be released. A lower detector located at either the gas collection chamber or the gas vent chamber, and an upper detector located at either the gas collection chamber or the gas vent chamber are provided. The lower detector is located below the upper detector. The lower and upper detectors are capable of detecting gas and liquid. A clamp is provided in the gas vent chamber either between the lower and the upper level detectors or above both level detectors. The apparatus also includes a control apparatus for opening and closing the clamp in response to whether the lower and upper detectors detect gas or liquid within the chamber.

In another embodiment of this invention, a method for venting a gas contained in a liquid flowing in a liquid flow circuit includes flowing liquid into a gas collection chamber located within the liquid flow circuit so that liquid flows through the gas collection chamber allowing gas to separate from the liquid and establish a gas-liquid interface within the gas collection chamber, detecting whether liquid is present at a lower position in either the gas collection chamber or a gas vent chamber by a lower level detector for detecting gas and liquid, opening a clamp if a liquid is not present at the lower position, detecting whether liquid is present at an upper position in either the gas collection chamber or the gas vent chamber by an upper level detector for detecting gas and liquid, and closing the clamp if liquid is present at the upper position.

In another embodiment of this invention, an extracorporeal hemodialysis circuit includes arterial tubing for receiving unfiltered blood from a patient, venous tubing for providing filtered blood to a patient, a dialyzer, and an apparatus for venting gas contained in a liquid. The dialyzer and apparatus for venting gas are located within the extracorporeal hemodialysis circuit so that blood flows from the patient, through the arterial tubing, through the dialyzer, through the apparatus for venting gas, and towards the venous tubing.

### BRIEF DESCRIPTION OF THE DRAWINGS

FIG. 1 is a schematic diagram of an extracorporeal liquid circuit illustrating a hemodialysis system.

FIG. 2 is a side view of a chamber for venting gas having two level detectors and a clamp.

FIG. 3 is a side view of a hemodialysis cassette for venting gas having two level detectors and a clamp.

FIG. 4 is flowchart of a method for venting gas in a system having two level detectors and a clamp.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

### DETAILED DESCRIPTION OF THE INVENTION

A description of example embodiments of the invention follows.

### Extracorporeal Circuit

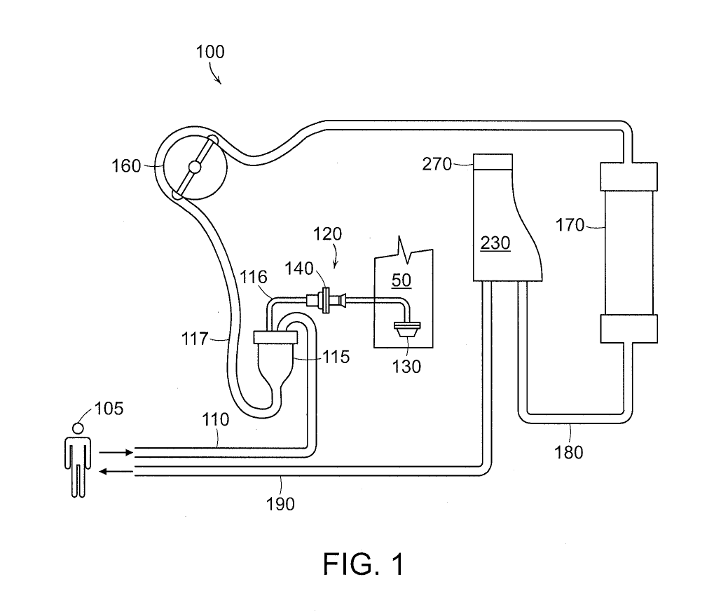

FIG. 1 illustrates a typical extracorporeal hemodialysis circuit 100, which includes tubing through which the blood flows and components for filtering and performing dialysis on the blood. Blood flows from a patient 105 through arterial tubing 110. After exiting the patient, blood drips into a drip chamber 115 where a connecting tube 116 from the drip chamber 115 attaches to an arterial pressure sensor assembly 120 that determines the pressure of the blood on the arterial side of the circuit 100.

A pump 160, such as a peristaltic pump, forces the blood to continue along the path through the circuit 100. After exiting the drip chamber 115, the blood then flows through tubing 117 to a dialyzer 170, which separates waste products from the blood. After passing through the dialyzer 170, the blood flows through venous tubing 180 towards a gas venting chamber 230 in which gas (e.g., air) in the blood can escape before the blood continues to the patient 105. After leaving the chamber 230, the blood travels through a venous line 190 and back to the patient 105. The gas collection apparatus and cassette subsequently described herein can be used with an extracorporeal hemodialysis circuit and device, as illustrated in FIG. 1.

### Gas Collection Apparatus and Cassette

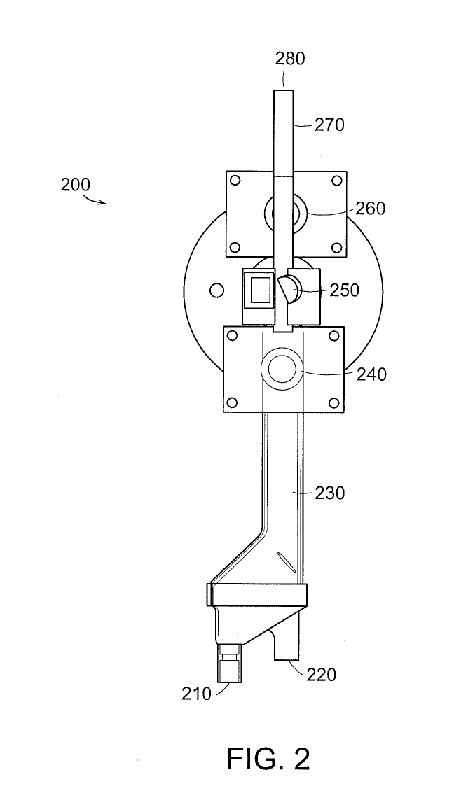

FIG. 2 illustrates an exemplary embodiment of a gas venting apparatus 200 having a chamber, two level detectors, and a clamp. The gas venting apparatus 200 has a liquid inlet 210 and a liquid outlet 220. In FIG. 2, the liquid inlet 210 is positioned below the liquid outlet 220, but the liquid inlet 210 can also be positioned above the liquid outlet 220 or at approximately the same height as the liquid outlet 220. A liquid, such as blood, enters through the liquid inlet 210 and leaves through the liquid outlet 220. The liquid can fill the volume of the gas collection chamber 230.

The lower level detector 240 and the upper level detector 260 can detect the presence of a gas or a liquid. The clamp 250 can open or close based on signals from the lower level detector 240 and the upper level detector 260. During operation, the gas collection chamber initially fills with a liquid, such as blood. The liquid can contain gas bubbles. Over time, the gas bubbles rise to the surface and begin to fill the gas collection chamber with the gas, thereby creating an interface between the gas and the liquid. As gas bubbles continue to rise to the surface, the interface between the gas and the liquid moves vertically down the gas collection chamber.

When the lower level detector 240 detects the presence of a liquid, the clamp 250 remains closed. When the gas-liquid interface crosses the location where the lower level detector is positioned, the level detector can send a signal indicative of the presence of a gas. The signal can be sent from the lower level detector to a control apparatus (not shown) that receives the signal. Upon receiving the signal, the control apparatus can send a signal to the clamp instructing the clamp to open.

Once the clamp opens, the gas in the gas collection chamber 230 can travel through the gas venting tube 270. The gas venting tube 270 has a gas outlet 280. In some embodiments, the outlet can vent gas to the atmosphere. When the clamp is open, the gas collection chamber 230 is in fluid communication with the gas vent tube 270, which in turn is in fluid communication with the atmosphere.

Ordinarily, the pressure in the gas collection chamber is greater than atmospheric pressure. Thus when the clamp 250 is open, the gas-liquid interface moves vertically up the gas collection chamber, which expunges accumulated gas to the atmosphere.

Similar to the lower level detector, the upper level detector 260 detects the presence of a gas or a liquid. When the clamp 250 is open, the gas-liquid interface can move vertically up the chamber and can cross the location where the upper level detector is positioned. When the upper level detector 260 detects the presence of a gas, the clamp can remain open, thus permitting further venting of gas to the atmosphere. When the upper level detector 260 detects the presence of a liquid, the clamp 250 can close, thus preventing the liquid from reaching the gas outlet 280. In some embodiments, the upper level detector can send a signal indicative of the presence of a gas or a liquid to the control apparatus (not shown). The control apparatus can then send a signal to the clamp that opens or closes the clamp.

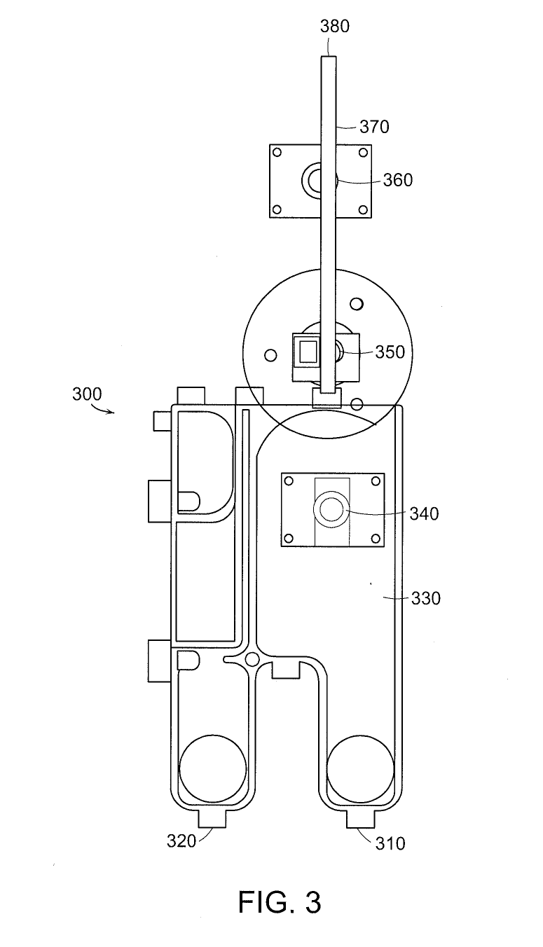

FIG. 3 illustrates a gas venting cassette 300 having a chamber, two level detectors, and a clamp. FIG. 3 is similar to FIG. 2, except that FIG. 3 is a cassette 300 including a liquid inlet 310, a liquid outlet 320, a gas collection chamber 330, a lower level detector 340, a clamp 350, an upper level detector 360, a gas vent tube 370, and a gas outlet 380. The embodiment of FIG. 3 operates similarly to the embodiment of FIG. 2. The primary difference between the embodiments of FIG. 2 and FIG. 3 is in the shape of the gas collection chamber.

The term “clamp” is used in its broadest sense, meaning that it is an element that is capable of opening and closing the gas vent tube. In one embodiment, the clamps (250 and 350) can be pinch clamps that, in a closed position, exert pressure on a tube to prevent the passage of gas or liquid. In another embodiment, the clamps (250 and 350) can be balloon clamps. A wide variety of suitable devices that can open and close the gas vent tube in response to a signal can be used.

In one embodiment, the level detectors (240, 260, 340, and 360) can detect the density of a substance. Liquids generally have a higher density than gasses. Thus, the level detector can send a signal indicative of the density of a substance, wherein the density is indicative of the presence of a gas or a liquid. In some embodiments, the liquid can be blood. In some embodiments, the gas can be air.

The gas bubbles can be dissolved in the liquid, or the gas bubbles can be too large to be considered dissolved in the liquid. In some cases, the gas bubbles can be observable with the naked eye. In other cases, the gas bubbles can on the order of magnitude of a millimeter or less.

The gas collection chamber (230 and 330) and the gas vent tube (270 and 370) are not necessarily separate pieces. Rather, the two can be an integrated component. In other words, the gas collection chamber and the gas vent tube can be a single, integral unit. While FIGS. 2 and 3 illustrate the lower level detector (240 and 340) positioned at the gas collection chamber (230 and 330) and the upper level detector (260 and 360) positioned at the gas vent chamber (270 and 370), the lower and upper level detectors (240, 260, 340, and 360) can be positioned at either the gas collection chamber (230 and 330) or at the gas vent tube (270 and 370).

Typically, the gas outlet (280 and 380) is capped with a hydrophobic membrane, such as a polytetrafluoroethylene (PTFE) or polyethylene (PE) membrane, though other suitable hydrophobic membranes can also be used.

The chamber embodiment of FIG. 2 and the cassette embodiment of FIG. 3 can be made of a wide variety of materials suitable for medical applications, and can be formed into the appropriate shape any processes suitable for medial applications.

Typically, the liquid inlet (210 and 310) and the liquid outlet (220 and 320) connect to tubing. As shown in FIG. 1, the liquid inlet (210 and 310) connects to tubing 180, and the liquid outlet (220 and 320) connect to tubing 190. The tubing can be of a wide variety of materials suitable for medical application.

### Operation of the Gas Collection Apparatus and Cassette

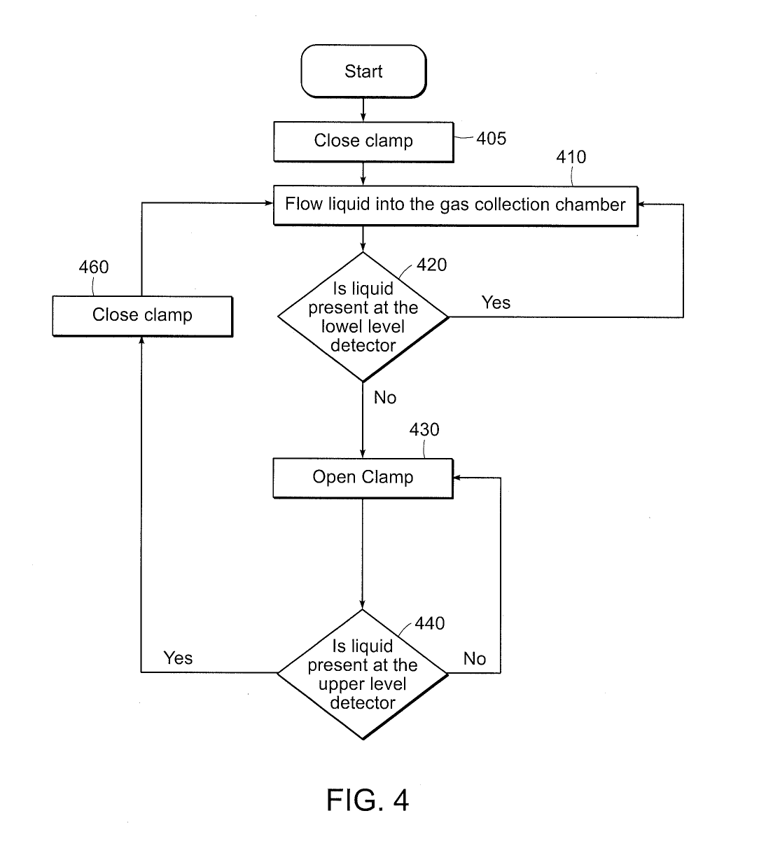

FIG. 4 is a flowchart illustrating steps in a method for venting gas in a system having a gas collection chamber, two level detectors, and a clamp. In one embodiment, the clamp is initially in a closed position. Optionally, the clamp can be closed if it is open (step 405). Liquid flows into the gas collection chamber (step 410). The liquid enters the gas collection chamber via a liquid inlet, such as liquid inlet 210 or 310. The lower level detector detects the presence of a liquid or gas (step 420). If liquid is present at the lower level detector (i.e., if gas is not present), the clamp remains closed and liquid continues to flow into the gas collection chamber (step 410). If liquid is not present at the lower level detector (i.e., if gas is present at the lower level detector), the clamp opens (step 430).

While the clamp is open, gas in the gas collection chamber is in fluid communication with the atmosphere via a gas venting tube, and gas can vent to the atmosphere. As gas vents to the atmosphere, the gas-liquid interface rises. The upper level detector detects the presence of a liquid or a gas (step 440). If liquid is not present at the upper level detector (i.e., if gas is present), the clamp remains open (step 430), and the upper level detector continues to detect the presence of liquid or gas (step 440). If liquid is present at the upper level detector (i.e., if gas is not present), then the clamp closes (step 460). Liquid continues to flow into the gas collection chamber (step 410) and the cycle repeats.

While FIGS. 4 describes detecting liquid at the upper level detector at step 440, the upper level detector can detect the presence of liquid or gas at the outset. However, the system and method do not need to consider the presence or absence of gas or liquid at the upper level detector until the clamp has opened.

While steps 420 and 440 described detecting whether liquid is present at the level detectors, it is equivalent to detect whether gas is present. In such case, the relative placement of the “Yes” and “No” answers to the inquiry are reversed. In other words, if gas is present at the lower level detector (step 420), then the clamp opens (step 430), and if gas is not present, then the clamp remains closed (step 410). Similarly, if gas is present at the upper level detector (step 440), then the clamp remains open (step 430), and if gas is not present, then the clamp closes (step 460).

While this invention has been particularly shown and described with references to example embodiments thereof, it will be understood by those skilled in the art that various changes in form and details may be made therein without departing from the scope of the invention encompassed by the appended claims.

What is claimed is:

1. An apparatus for venting gas contained in a liquid flowing in a liquid flow circuit comprising:
a) a gas collection chamber located within the liquid flow circuit so that liquid flows through the gas collection chamber allowing gas to separate from the liquid and establish a gas-liquid interface within the gas collection chamber;
b) a gas vent chamber at the top of the gas collection chamber through which gas within the gas collection chamber can be released;
c) a lower detector located in either the gas collection chamber or the gas vent chamber, and an upper detector located in either the gas collection chamber or the gas vent chamber, said lower detector located below said upper detector, and said lower and upper detectors being capable of detecting gas or liquid;
d) a clamp located in said gas vent chamber either between the lower and the upper level detectors or above both level detectors; and
e) a control apparatus for opening and closing the clamp in response to whether the lower and upper detectors detect gas or liquid within the gas collection chamber.

2. The apparatus of claim 1, wherein the liquid is blood.

3. The apparatus of claim 2, wherein the clamp is a pinch clamp.

4. The apparatus of claim 2, wherein the clamp is a balloon clamp.

5. The apparatus of claim 2, wherein the lower level detector can detect density.

6. The apparatus of claim 5, wherein the upper level detector can detect density.

7. The apparatus of claim 6, wherein the apparatus is a cassette.

8. The apparatus of claim 1, wherein the gas collection chamber and the gas vent chamber are a single unit.

9. A method for venting gas contained in a liquid flowing in a liquid flow circuit comprising:
flowing the liquid into a gas collection chamber located within the liquid flow circuit so that liquid flows through the gas collection chamber allowing gas to separate from the liquid and establish a gas-liquid interface within the gas collection chamber;
detecting whether liquid is present at a lower position in either the gas collection chamber or a gas vent chamber by a lower level detector;
opening a clamp if a liquid is not present at the lower position;
detecting whether liquid is present at an upper position in either the gas collection chamber or the gas vent chamber by an upper level detector; and
closing the clamp if liquid is present at the upper position.

10. An extracorporeal hemodialysis circuit comprising:
a) arterial tubing for receiving unfiltered blood from a patient;
b) venous tubing for providing filtered blood to a patient; and
c) a dialyzer and an apparatus for venting gas according to claim 1 located within the extracorporeal hemodialysis circuit so that blood flows from the patient, through the arterial tubing, through the dialyzer, through the apparatus for venting gas according to claim 1, and towards the venous tubing.
